(19) United States
(12) Patent Application Publication
(10) Pub. No.: US 2003/0008814 A1
(43) Pub. Date: Jan. 9, 2003

(54) Pseudomycin phosphate prodrugs

(76) Inventors: Shu Hui Chen, Carmel, IN (US); Xicheng David Sun, Superior, CO (US); Yanzhi Zhang, Sharon, MA (US)

Correspondence Address: ELI LILLY AND COMPANY, PATENT DIVISION, P.O. BOX 6288, INDIANAPOLIS, IN, 46206-6288, US

(21) Appl. No.: 10/130,835

(22) PCT Filed: Nov. 29, 2000
(86) PCT No.: PCT/US00/30167

(51) Int. Cl. A61K 38/12; C07K 7/50

(52) U.S. Cl. 514/9; 530/317

(57) ABSTRACT

A pseudomycin prodrug represented by structure (A) where R1 is a phosphate benzyloxycarbamate or phosphate methyleneoxycarbamate linkage is described. The phosphate prodrug demonstrates antifungal activity with less adverse side effects than the parent pseudomycin compound.

### FIELD OF THE INVENTION

The present invention relates to pseudomycin compounds, in particular, phosphate prodrugs of pseudomycin compounds.

### BACKGROUND OF THE INVENTION

Pseudomycins are natural products isolated from liquid cultures of Pseudomonas syringae (plant-associated bacterium) and have been shown to have antifungal activities. (see i.e., Harrison, L., et al., “Pseudomycins, a family of novel peptides from Pseudomonas syringae possessing broad-spectrum antifungal activity,” J. Gen. Microbiology, 137(12), 2857-65 (1991) and U.S. Pat. Nos. 5,576,298 and 5,837,685) Unlike the previously described antimycotics from P. syringae (e.g., syringomycins, syringotoxins and syringostatins), pseudomycins A-C contain hydroxyaspartic acid, aspartic acid, serine, dehydroaminobutyric acid, lysine and diaminobutyric acid. The peptide moiety for pseudomycins A, A′, B, B′, C, C′ corresponds to L-Ser-D-Dab-L-Asp-L-Lys-L-Dab-L-aThr-Z-Dhb-L-Asp(3-OH)-L-Thr(4-Cl) with the terminal carboxyl group closing a macrocyclic ring on the OH group of the N-terminal Ser. The analogs are distinguished by the N-acyl side chain, i.e., pseudomycin A is N-acylated by 3,4-dihydroxytetradecanoate, pseudomycin A′ by 3,4-dihydroxypentadecanoate, pseudomycin B by 3-hydroxytetradecanoate, pseudomycin B′ by 3-hydroxydodecanoate, pseudomycin C by 3,4-dihydroxyhexadecanoate and pseudomycin C′ by 3-hydroxyhexadecanoate. (see i.e., Ballio, A., et al., “Novel bioactive lipodepsipeptides from Pseudomonas syringae: the pseudomycins,” FEBS Letters, 355(1), 96-100, (1994) and Coiro, V. M., et al., “Solution conformation of the Pseudomonas syringae MSU 16H phytotoxic lipodepsipeptide Pseudomycin A determined by computer simulations using distance geometry and molecular dynamics from NMR data,” Eur. J. Biochem., 257(2), 449-456 (1998).)

Pseudomycins are known to have certain adverse biological effects. For example, destruction of the endothelium of the vein, destruction of tissue, inflammation, and local toxicity to host tissues have been observed when pseudomycin is administered intraveneously. Therefore, there is a need to identify compounds within this class that are useful for treating fungal infections without the currently observed adverse side effects.

### BRIEF SUMMARY OF THE INVENTION

The present invention provides a pseudomycin prodrug represented by the following structure which is useful as an antifungal agent.

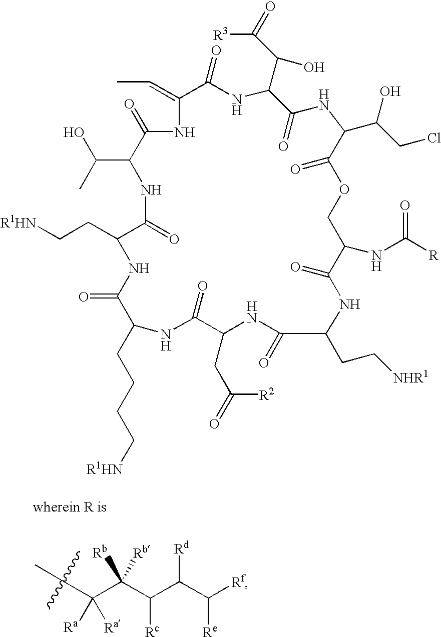

where

R^{a} and R^{a′} are independently hydrogen or methyl, or either R^{a} or R^{a}′ is alkyl amino, taken together with R^{b} or R^{b}′ forms a six-membered cycloalkyl ring, a six-membered aromatic ring or a double bond, or taken together with R^{c} forms a six-membered aromatic ring;

R^{b} and R^{b}′ are independently hydrogen, halogen, or methyl, or either R^{b} or R^{b}′ is amino, alkylamino, α-acetoacetate, methoxy, or hydroxy;

R^{c} is hydrogen, hydroxy, C₁-C₄ alkoxy, hydroxy C₁-C₄ alkoxy, or taken together with R^{e} forms a 6-membered aromatic ring or C₅-C₆ cycloalkyl ring;

R^{d} is hydrogen;

R^{e} is hydrogen, or taken together with R^{f} is a six-membered aromatic ring, C₅-C₁₄ alkoxy substituted six-membered aromatic ring, or C₅-C₁₄ alkyl substituted six-membered aromatic ring, and

R^{f} is C₈-C₁₈ alkyl, C₅-C₁₁ alkoxy, or biphenyl;

or R is

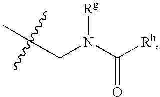

where

R^{g} is hydrogen, or C₁-C₁₃ alkyl, and

R^{h} is C₁-C₁₅ alkyl, C₄-C₁₅ alkoxy, (C₁-C₁₀ alkyl)phenyl, —(CH₂)ₙ-aryl, or —(CH₂)ₙ—(C₅-C₆ cycloalkyl), where n=1 or 2; or

R is

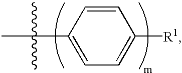

where

Rⁱ is a hydrogen, halogen, or C₅-C₈ alkoxy, and

m is 1, 2 or 3:

or R is

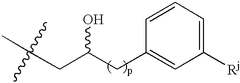

where

R^{j} is C₅-C₁₄ alkoxy or C₅-C₁₄ alkyl, and p=0, 1 or 2,

or R is

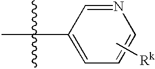

where

R^{k} is C₅-C₁₄ alkoxy; or

R is —(C₂)—NR^{m}—(C₁₃-C₁₈ alkyl), where R^{m} is H, —CH₃ or —C(O)CH₃;

R^{l} is independently hydrogen or a group represented by formula 1(a), 1(b), or 1(c)

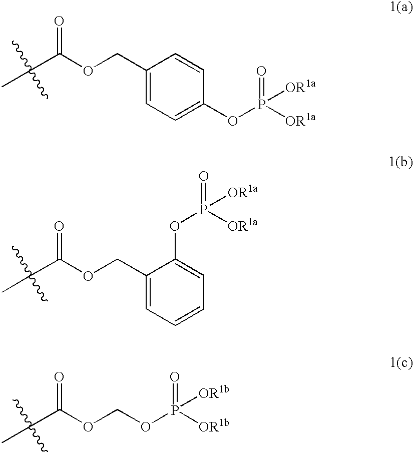

where

R^{1a} is hydrogen, C₁-C₆ alkyl (e.g., methyl, ethyl, n-propyl, i-propyl, n-butyl, i-butyl, s-butyl, t-butyl, etc.), benzyl, or —CH₂CH₂Si(CH₃)₃, and

R^{1b} is hydrogen or C₁-C₆ alkyl, provided that at least one R¹ is a group represented by formula 1(a), 1(b) or 1(c);

R² and R³ are independently —OR^{2a}, or —N(R^{2b})(R^{2c})

where

R^{2a} and R^{2b} are independently hydrogen, C₁-C₁₀ alkyl (e.g., methyl, ethyl, n-propyl, i-propyl, n-butyl, i-butyl, s-butyl, t-butyl, etc.), C₃-C₆ cycloalkyl (e.g., cyclopropyl, cyclobutyl, cyclopentyl, cyclopentylmethylene, methylcyclopentyl, cyclohexyl, etc.) hydroxy(C₁-C₁₀)alkyl, alkoxy(C₁-C₁₀)alkyl (e.g, methoxyethyl), or C₂-C₁₀ alkenyl, amino(C₁-C₁₀)alkyl, mono- or di-alkylamino(C₁-C₁₀ )alkyl, aryl (C₁-C₁₀ )alkyl (e.g., benzyl), heteroaryl(C₂-C₁₀)alkyl (e.g., 3-pyridylmethyl, 4-pyridylmethyl), or cycloheteroalkyl(C₁-C₁₀ )alkyl (e.g., N-tetrahydro-1,4-oxazinylethyl and N-piperazinylethyl), or

R^{2b} is an alkyl carboxylate residue of an aminoacid alkyl ester (e.g., —CH₂CO₂CH₃, —CH(CO₂CH₃)CH(CH₃)₂, —CH(CO₂CH₃)CH(phenyl), —CH(CO₂CH₃)CH₂OH, —CH(CO₂CH₃)CH₂(p-hydroxyphenyl), —CH (CO₂CH₃)CH₂SH, —CH(CO₂CH₃)CH₂(CH₂)₃NH₂, —CH(CO₂CH₃)CH₂(4- or 5-imidazole), —CH(CO₂CH₃)CH₂CO₂CH₃, —CH(CO₂CH₃)CH₂CO₂NH₂, and the like), and

R^{2c} is hydrogen or C₁-C₆ alkyl; and pharmaceutically acceptable salts and solvates thereof. In a preferred embodiment, R is represented by the structure

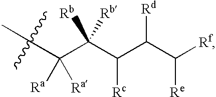

where R^{b}′ is hydroxy, R^{a}, R^{a}′, R^{b}, R^{c}, R^{d}, and R^{e} are all hydrogen, and R^{f} is n-octyl.

In another embodiment of the present invention, a pharmaceutical formulation is provided which includes the pseudomycin prodrug described above and a pharmaceutically acceptable carrier.

In yet another embodiment of the present invention, a method is provided for treating a fungal infection in an animal in need thereof, which comprises administering to the animal the pseudomycin prodrug described above. The use of the pseudomycin prodrug described above in the manufacture of a medicament for use in treating a fungal infection in an animal is also provided.

### Definitions

As used herein, the term “alkyl” refers to a hydrocarbon radical of the general formula CₙH₂ₙ₊₁ containing from 1 to 30 carbon atoms unless otherwise indicated. The alkane radical may be straight (e.g. methyl, ethyl, propyl, butyl, etc.), branched (e.g , isopropyl, isobutyl, tertiary butyl, neopentyl, etc.), cyclic (e.g., cyclopropyl, cyclobutyl, cyclopentyl, methylcyclopentyl, cyclohexyl, etc.), or multi-cyclic (e.g., bicyclo[2.2.1]heptane, spiro[2.2]pentane, etc.). The alkane radical may be substituted or unsubstituted. Similarly, the alkyl portion of an alkoxy group, alkanoyl, or alkanoate have the same definition as above.

The term “alkenyl” refers to an acyclic hydrocarbon containing at least one carbon carbon double bond. The alkene radical may be straight, branched, cyclic, or multi-cyclic. The alkene radical may be substituted or unsubstituted. The alkenyl portion of an alkenoxy, alkenoyl or alkenoate group has the same definition as above.

The term “aryl” refers to aromatic moieties having single (e.g., phenyl) or fused ring systems (e.g., naphthalene, anthracene, phenanthrene, etc.). The aryl groups may be substituted or unsubstituted.

Within the field of organic chemistry and particularly within the field of organic biochemistry, it is widely understood that significant substitution of compounds is tolerated or even useful. In the present invention, for example, the term alkyl group allows for substitutents which is a classic alkyl, such as methyl, ethyl, propyl, hexyl, isooctyl, dodecyl, stearyl, etc. The term “group” specifically envisions and allows for substitutions on alkyls which are common in the art, such as hydroxy, halogen, alkoxy, carbonyl, keto, ester, carbamato, etc., as well as including the unsubstituted alkyl moiety. However, it is generally understood by those skilled in the art that the substituents should be selected so as to not adversely affect the pharmacological characteristics of the compound or adversely interfere with the use of the medicament. Suitable substituents for any of the groups defined above include alkyl, alkenyl, alkynyl, aryl, halo, hydroxy, alkoxy, aryloxy, mercapto, alkylthio, arylthio, mono- and di-alkyl amino, quaternary ammonium salts, aminoalkoxy, hydroxyalkylamino, aminoalkylthio, carbamyl, carbonyl, carboxy, glycolyl, glycyl, hydrazino, guanyl, and combinations thereof.

The term “prodrug” refers to a class of drugs which result in pharmacological action due to conversion by metabolic processes within the body (i.e., biotransformation). In the present invention, the pseudomycin prodrug compounds contain linkers that can be cleaved by esterases in the plasma to produce the active drug.

The term “animal” refers to humans, companion animals (e.g., dogs, cats and horses), food-source animals (e.g., cows, pigs, sheep and poultry), zoo animals, marine animals, birds and other similar animal species.

### DETAILED DESCRIPTION OF THE INVENTION

Applicants have discovered that a prodrug derivative of the pseudomycin natural or semi-synthetic products provide less adverse side effects than the corresponding natural products and maintains in vivo efficacy against C. albican, C. neoformans, and A. fumigatus. The prodrug is produced by acylating at least one of the pendant amino groups attached to the lysine or 2,4-diaminobutyric acid peptide units in the pseudomycin cyclopeptide ring system to form an acyl phosphate substituent(s). The phosphate acylating agent (or linker) is generally a compound having one of following formulae 1a-L, 1b-L or 1c-L

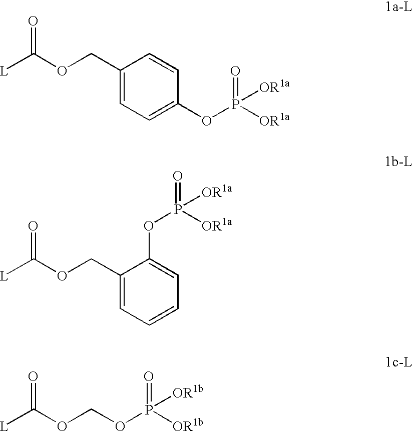

where L is a suitable leaving group such that a carbamate linkage with the pendant amino group on the pseudomycin structure can be formed. Suitable leaving groups are well known to those skilled in the art and include groups such as p-nitrophenoxy and N-oxysuccinimide. R^{1a} and R^{1b} are as defined above.

The benzyl phosphate linkers (1a-L) may be synthesized using the synthetic route shown in scheme I below. For illustrative purposes, a specific para-phenylmethylene substituted acylating compound having a p-nitrophenoxy leaving group is depicted. However, it will be understood by those skilled in the art that one could synthesize a variety of derivatives (including the ortho substituted derivative (1b-L)) using the same basic synthetic method.

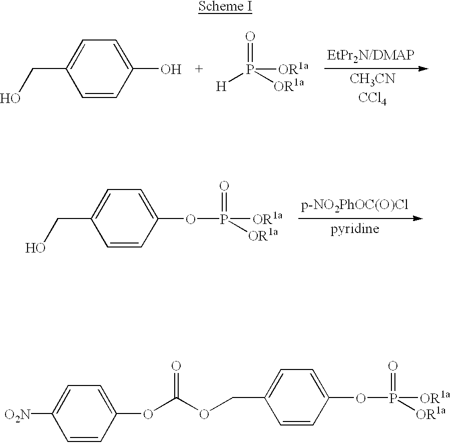

For a more detailed description of the synthetic procedures, see the preparation section of the Examples below.

The phosphate linker (1a-L or 1b-L) where R^{1a} is —CH₂CH₂Si(CH₃)₃ may be prepared by oxidative coupling of bis-teocphosphite with 4-hydroxybenzyl alcohol in carbon tetrachloride as described in Li, J., et al., Bioorg. Med. Chem. Lett., 8, 3159-3164 (1998). Bis-teocphosphite may be prepared from 2-2-trimethylsilylethanol and PCl₃ as described in McCombie, et al., J. Chem. Soc., 381 (1945).

The methylene phosphate linker (1c-L) may be synthesized using the synthetic route shown in scheme II below. For illustrative purposes, a specific acylating compound with an N-succinimide leaving group is depicted. However, it will be understood by those skilled in the art that one could synthesize a variety of derivatives using the same basic synthetic method.

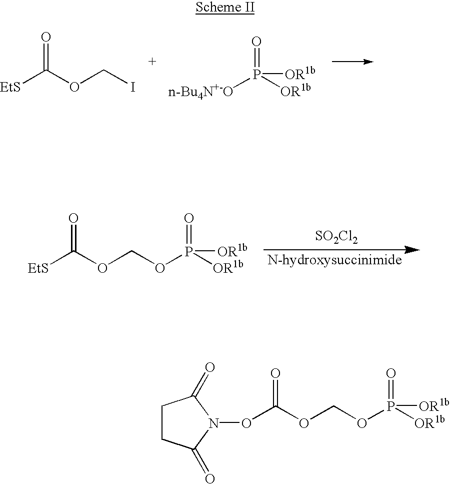

For a more detailed description of the synthetic procedures, see the preparation section of the Examples below. The phosphate triesters (methyleneoxy and benzyloxy derivatives) may be converted to their phosphate monoester derivatives via hydrogenation (e.g., hydrogen over Pd/C in methanol).

As discussed earlier, pseudomycins are natural products isolated from the bacterium Pseudomonas syringae that have been characterized as lipodepsinonapetpides containing a cyclic peptide portion closed by a lactone bond and including the unusual amino acids 4-chlorothreonine (ClThr), 3-hydroxyaspartic acid (HOAsp), 2,3-dehydro-2-aminobutyric acid (Dhb), and 2,4-diaminobutyric acid (Dab). Methods for growth of various strains of P. syringae to produce the different pseudomycin analogs (A, A′, B, B′, C, and C′) are described below and described in more detail in PCT Patent Application Serial No. PCT/US00/08728 filed by Hilton, et al. on April 14, 2000 entitled “Pseudomycin Production by Pseudomonas Syringae,” incorporated herein by reference, PCT Patent Application Serial No. PCT/US00/08727 filed by Kulanthaivel, et al. on Apr. 14, 2000 entitled “Pseudomycin Natural Products,” incorporated herein by reference, and U.S. Pat. Nos. 5,576,298 and 5,837,685, each of which are incorporated herein by reference.

Isolated strains of P. syringae that produce one or more pseudomycins are known in the art. Wild type strain MSU 174 and a mutant of this strain generated by transposon mutagenesis, MSU 16H (ATCC 67028) are described in U.S. Pat. Nos. 5,576,298 and 5,837,685; Harrison, et al., “Pseudomycins, a family of novel peptides from Pseudomonas syringae possessing broad-spectrum antifungal activity,” J. Gen. Microbiology, 137, 2857-2865 (1991); and Lamb et al., “Transposon mutagenesis and tagging of fluorescent pseudomonas: Antimycotic production is necessary for control of Dutch elm disease,” Proc. Natl. Acad. Sci. USA, 84, 6447-6451 (1987).

A strain of P. syringae that is suitable for production of one or more pseudomycins can be isolated from environmental sources including plants (e.g., barley plants, citrus plants, and lilac plants) as well as, sources such as soil, water, air, and dust. A preferred stain is isolated from plants. Strains of P. syringae that are isolated from environmental sources can be referred to as wild type. As used herein, “wild type” refers to a dominant genotype which naturally occurs in the normal population of P. syringae (e.g., strains or isolates of P. syringae that are found in nature and not produced by laboratory manipulation). Like most organisms, the characteristics of the pseudomycin-producing cultures employed (P. syringae strains such as MSU 174, MSU 16H, MSU 206, 25-B1, 7H9-1) are subject to variation. Hence, progeny of these strains (e.g., recombinants, mutants and variants) may be obtained by methods known in the art.

P. syringae MSU 16H is publicly available from the American Type Culture Collection, Parklawn Drive, Rockville, MD, USA as Accession No. ATCC 67028. P. syringae strains 25-B1, 7H9-1, and 67 H1 were deposited with the American Type Culture Collection on Mar. 23, 2000 and were assigned the following Accession Nos.:

| | |
|---|---|
| 25-B1 | Accession No. PTA-1622 |
| 7H9-1 | Accession No. PTA-1623 |
| 67 H1 | Accession No. PTA-1621 |

Mutant strains of P. syringae are also suitable for production of one or more pseudomycins. As used herein, “mutant” refers to a sudden heritable change in the phenotype of a strain, which can be spontaneous or induced by known mutagenic agents, such as radiation (e.g., ultraviolet radiation or x-rays), chemical mutagens (e.g., ethyl methanesulfonate (EMS), diepoxyoctane, N-methyl-N-nitro-N′-nitrosoguanine (NTG), and nitrous acid), site-specific mutagenesis, and transposon mediated mutagenesis. Pseudomycin-producing mutants of P. syringae can be produced by treating the bacteria with an amount of a mutagenic agent effective to produce mutants that overproduce one or more pseudomycins, that produce one pseudomycin (e.g., pseudomycin B) in excess over other pseudomycins, or that produce one or more pseudomycins under advantageous growth conditions. While the type and amount of mutagenic agent to be used can vary, a preferred method is to serially dilute NTG to levels ranging from 1 to 100 μg/ml. Preferred mutants are those that overproduce pseudomycin B and grow in minimal defined media.

Environmental isolates, mutant strains, and other desirable strains of P. syringae can be subjected to selection for desirable traits of growth habit, growth medium nutrient source, carbon source, growth conditions, amino acid requirements, and the like. Preferably, a pseudomycin producing strain of P. syringae is selected for growth on minimal defined medium such as N21 medium and/or for production of one or more pseudomycins at levels greater than about 10 μg/ml. Preferred strains exhibit the characteristic of producing one or more pseudomycins when grown on a medium including three or fewer amino acids and optionally, either a lipid, a potato product or combination thereof.

Recombinant strains can be developed by transforming the P. syringae strains, using procedures known in the art. Through the use of recombinant DNA technology, the P. syringae strains can be transformed to express a variety of gene products in addition to the antibiotics these strains produce. For example, one can modify the strains to introduce multiple copies of the endogenous pseudomycin-biosynthesis genes to achieve greater pseudomycin yield.

To produce one or more pseudomycins from a wild type or mutant strain of P. syringae, the organism is cultured with agitation in an aqueous nutrient medium including an effective amount of three or fewer amino acids, preferably glutamic acid, glycine, histidine, or a combination thereof. Alternatively, glycine is combined with one or more of a potato product and a lipid. Culturing is conducted under conditions effective for growth of P. syringae and production of the desired pseudomycin or pseudomycins. Effective conditions include temperatures from about 22° C. to about 27° C., and a duration of about 36 hours to about 96 hours. Controlling the concentration of oxygen in the medium during culturing of P. syringae is advantageous for production of a pseudomycin. Preferably, oxygen levels are maintained at about 5 to 50% saturation, more preferably about 30% saturation. Sparging with air, pure oxygen, or gas mixtures including oxygen can regulate the concentration of oxygen in the medium.

Controlling the pH of the medium during culturing of P. syringae is also advantageous. Pseudomycins are labile at basic pH, and significant degradation can occur if the pH of the culture medium is above about 6 for more than about 12 hours. Preferably, the pH of the culture medium is maintained between 6 and 4. P. syringae can produce one or more pseudomycins when grown in batch culture. However, fed-bath or semi-continuous feed of glucose and optionally, an acid or base (e.g., ammonium hydroxide) to control pH, enhances production. Pseudomycin production can be further enhanced by using continuous culture methods in which glucose and ammonium hydroxide are fed automatically.

Choice of P. syringae strain can affect the amount and distribution of pseudomycin or pseudomycins produced. For example, strains MSU 16H and 67 H1 each produce predominantly pseudomycin A, but also produce pseudomycin B and C, typically in ratios of 4:2:1. Strain 67 H1 typically produces levels of pseudomycins about three to five fold larger than are produced by strain MSU 16H. Compared to strains MSU 16H and 67 H1, strain 25-B1 produces more pseudomycin B and less pseudomycin C. Strain 7H9-1 are distinctive in producing predominantly pseudomycin B and larger amount of pseudomycin B than other strains. For example, this strain can produce pseudomycin B in at least a ten fold excess over either pseudomycin A or C.

Alternatively, the prodrug can be formed from an N-acyl semi-synthetic compound. Semi-synthetic pseudomycin compounds may be synthesized by exchanging the N-acyl group on the L-serine unit. Examples of various N-acyl derivatives are described in PCT Application No. PCT/US00/15017 filed by Chen, et al. on Jun. 8, 2000 entitled “Pseudomycin N-Acyl Side-Chain Analogs” and incorporated herein by reference. In general, four synthetic steps are used to produce the semi-synthetic compounds from naturally occurring pseudomycin compounds: (1) selective amino protection; (2) chemical or enzymatic deacylation of the N-acyl side-chain; (3) reacylation with a different side-chain; and (4) deprotection of the amino groups.

The pendant amino groups at positions 2, 4 and 5 may be protected using any standard means known to those skilled in the art for amino protection- The exact genus and species of amino protecting group employed is not critical so long as the derivatized amino group is stable to the condition of subsequent reactions) on other positions of the intermediate molecule and the protecting group can be selectively removed at the appropriate point without disrupting the remainder of the molecule including any other amino protecting group(s). Suitable amino-protecting groups include benzyloxycarbonyl, p-nitrobenzyloxycarbonyl, p-bromobenzyloxycarbonyl, p-methoxybenxyloxycarbonyl, p-methoxyphenylazobenzyloxycarbonyl, p-phenylazobenzyloxycarbonyl, t-butyloxycarbonyl, cyclopentyloxycarbonyl, and phthalimido. Preferred amino protecting groups are t-butoxycarbonyl (t-Boc), allyloxycarbonyl (Alloc), phthalimido, and benzyloxycarbonyl (CbZ or CBZ). Further examples of suitable protecting groups are described in T.W. Greene, “Protective Groups in Organic Synthesis,” John Wiley and Sons, New York, N.Y., (2nd ed., 1991), at chapter 7.

The deacylation of a N-acyl group having a gamma or delta hydroxylated side chain (e.g., 3,4-dihydroxytetra-deconoate) may be accomplished by treating the amino-protected pseudomycin compound with acid in an aqueous solvent. Suitable acids include acetic acid and trifluoroacetic acid. A preferred acid is trifluoroacetic acid. If trifluoroacetic acid is used, the reaction may be accomplished at or near room temperature. However, when acetic acid is used the reaction is generally ran at about 40° C. Suitable aqueous solvent systems include acetonitrile, water, and mixtures thereof. Organic solvents accelerate the reaction; however, the addition of an organic solvent may lead to other by-products. Pseudomycin compounds lacking a delta or gamma hydroxy group on the side chain (e.g., Pseudomycin B and C′) may be deacylated enzymatically. Suitable deacylase enzymes include Polymyxin Acylase (164-16081 Fatty Acylase (crude) or 161-16091 Fatty Acylase (pure) available from Wako Pure Chemical Industries, Ltd.), or ECB deacylase. The enzymatic deacylation may be accomplished using standard deacylation procedures well known to those skilled in the art. For example, general procedures for using polymyxin acylase may be found in Yasuda, N., et al, Agric. Biol. Chem., 53, 3245 (1989) and Kimura, Y., et al., Agric. Biol. Chem., 53, 497 (1989).

The deacylated product (also known as the pseudomycin nucleus) is reacylated using the corresponding acid of the desired acyl group in the presence of a carbonyl activating agent. “Carbonyl activating group” refers to a substituent of a carbonyl that promotes nucleophilic addition reactions at that carbonyl. Suitable activating substituents are those which have a net electron withdrawing effect on the carbonyl. Such groups include, but are not limited to, alkoxy, aryloxy, nitrogen containing aromatic heterocycles, or amino groups (e.g., oxybenzotriazole, imidazolyl, nitrophenoxy, pentachlorophenoxy, N-oxysuccinimide, N,N′-dicyclohexylisoure-O-yl, and N-hydroxy-N-methoxyamino); acetates; formates; sulfonates (e.g., methanesulfonate, ethanesulfonate, benzenesulfonate, and p-tolylsulfonate); and halides (e.g., chloride, bromide, and iodide).

A variety of acids may be used in the acylation process. Suitable acids include aliphatic acids containing one or more pendant aryl, alkyl, amino(including primary-secondary and tertiary amines), hydroxy, alkoxy, and amido groups; aliphatic acids containing nitrogen or oxygen within the aliphatic chain; aromatic acids substituted with alkyl, hydroxy, alkoxy and/or alkyl amino groups; and heteroaromatic acids substituted with alkyl, hydroxy, alkoxy and/or alkyl amino groups.

Alternatively, a solid phase synthesis may be used where a hydroxybenzotriazole-resin (HOBt-resin) serves as the coupling agent for the acylation reaction.

Once the amino group is deacylated and reacylated (described above), then the amino protecting groups (at positions 2, 4 and 5) can be removed by hydrogenation in the presence of a hydrogenation catalyst (e.g., 10% Pd/C). When the amino protecting group is allyloxycarbonyl, then the protecting group can be removed using tributyltinhydride and triphenylphosphine palladium dichloride. This particular protection/deprotection scheme has the advantage of reducing the potential for hydrogenating the vinyl group of the Z-Dhb unit of the pseudomycin structure.

The prodrug is then produced by acylating at least one of the pendant amino groups attached to the lysine or 2,4-diaminobutyric acid peptide units of the N-acyl modified semi-synthetic pseudomycin compound to form the desired carbamate linkage.

Other modified prodrug pseudomycin compounds may be synthesized by amidation or esterification of the pendant carboxylic acid group of the aspartic acid and/or hydroxyaspartic acid units of the pseudomycin ring. Examples of various acid-modified derivatives are described in PCT Application No. PCT/US00/15021 filed by Chen, et al. on Jun. 8, 2000 entitled “Pseudomycin Amide & Ester Analogs” and incorporated herein by reference. The acid-modified derivatives may be formed by condensing any of the previously described prodrugs with the appropriate alcohol or amine to produce the respective ester or amide.

Formation of the ester groups may be accomplished using standard esterification procedures well-known to those skilled in the art. Esterification under acidic conditions typically includes dissolving or suspending the pseudomycin compound in the appropriate alcohol in the presence of a protic acid (e.g., HCl, TFA, etc.). Under basic conditions, the pseudomycin compound is generally reacted with the appropriate alkyl halide in the presence of a weak base (e.g., sodium bicarbonate and potassium carbonate).

Formation of the amide groups may be accomplished using standard amidation procedures well-known to those skilled in the art. However, the choice of coupling agents provides selective modification of the acid groups. For example, the use of benzotriazol-1-yloxy-tripyrrolidinophosphonium hexafluorophosphate(PyBOP) as the coupling agent allows one to isolate pure mono-amides at residue 8 and (in some cases) pure bis amides simultaneously. Whereas, the use of o-benzotriazol-1-yl-N,N,N′,N′-tetramethyluronium tetrafluoroborate (TBTU) as the coupling agent favors formation of monoamides at residue 3.

The pseudomycin prodrug may be isolated and used per se or in the form of its pharmaceutically acceptable salt or solvate. The prodrug is prepared by forming at least one phosphate carbamate linkage as described earlier. The term “pharmaceutically acceptable salt” refers to non-toxic acid addition salts derived from inorganic and organic acids. Suitable salt derivatives include halides, thiocyanates, sulfates, bisulfates, sulfites, bisulfites, arylsulfonates, alkylsulfates, phosphonates, monohydrogen-phosphates, dihydrogenphosphates, metaphosphates, pyrophosphonates, alkanoates, cycloalkylalkanoates, arylalkonates, adipates, alginates, aspartates, benzoates, fumarates, glucoheptanoates, glycerophosphates, lactates, maleates, nicotinates, oxalates, palmitates, pectinates, picrates, pivalates, succinates, tartarates, citrates, camphorates, camphorsulfonates, digluconates, trifluoroacetates, and the like.

The term “solvate” refers to an aggregate that comprises one or more molecules of the solute (i.e., pseudomycin prodrug compound) with one or more molecules of a pharmaceutical solvent, such as water, ethanol, and the like. When the solvent is water, then the aggregate is referred to as a hydrate. Solvates are generally formed by dissolving the prodrug in the appropriate solvent with heat and slowing cooling to generate an amorphous or crystalline solvate form.

Each pseudomycin, semi-synthetic pseudomycin, pseudomycin prodrug and mixtures can be detected, determined, isolated, and/or purified by any variety of methods known to those skilled in the art. For example, the level of pseudomycin or pseudomycin prodrug activity in a broth or in an isolate or purified composition can be determined by antifungal action against a fungus such as Candida and can be isolated and purified by high performance liquid chromatography.

The active ingredient (i.e., pseudomycin derivative) is typically formulated into pharmaceutical dosage forms to provide an easily controllable dosage of the drug and to give the physician, patient, or veterinarian an elegant and easily handleable product. Formulations may comprise from 0.1% to 99.9% by weight of active ingredient, more generally from about 10% to about 30% by weight.

As used herein, the term “unit dose” or “unit dosage” refers to physically discrete units that contain a predetermined quantity of active ingredient calculated to produce a desired therapeutic effect. When a unit dose is administered orally or parenterally, it is typically provided in the form of a tablet, capsule, pill, powder packet, topical composition, suppository, wafer, measured units in ampoules or in multidose containers, etc. Alternatively, a unit dose may be administered in the form of a dry or liquid aerosol which may be inhaled or sprayed.

The dosage to be administered may vary depending upon the physical characteristics of the animal, the severity of the animal's symptoms, and the means used to administer the drug. The specific dose for a given animal is usually set by the judgment of the attending physician or veterinarian.

Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the active ingredient is being applied. The formulations may also include wetting agents, lubricating agents, surfactants, buffers, tonicity agents, bulking agents, stabilizers, emulsifiers, suspending agents, preservatives, sweeteners, perfuming agents, flavoring agents and combinations thereof.

A pharmaceutical composition may be administered using a variety of methods. Suitable methods include topical (e.g., ointments or sprays), oral, injection and inhalation. The particular treatment method used will depend upon the type of infection being addressed.

In parenteral iv applications, the formulations are typically diluted or reconstituted (if freeze-dried) and further diluted if necessary, prior to administration. An example of reconstitution instructions for the freeze-dried product are to add ten ml of water for injection (WFI) to the vial and gently agitate to dissolve. Typical reconstitution times are less than one minute. The resulting solution is then further diluted in an infusion solution such as dextrose 5% in water (D5W), prior to administration.

Pseudomycin compounds have been shown to exhibit antifungal activity such as growth inhibition of various infectious fungi including Candida spp. (i.e., C. Albicans, C. Parapsilosis, C. Krusei, C. Glabrata, C. Tropicalis, or C. Lusitaniaw); Torulopus spp.(i.e., T. Glabrata); Aspergillus spp. (i.e., A. Fumigatus); Histoplasma spp. (i.e., H. Capsulatum); Cryptococcus spp. (i.e., C. Neoformans); Blastomyces spp. (i.e., B. Dermatitidis); Fusarium spp.; Trichophyton spp., Pseudallescheria boydii, Coccidioides immits, Sporothrix schenckii, etc.

Consequently, the compounds and formulations of the present invention are useful in the preparation of medicaments for use in combating either systemic fungal infections or fungal skin infections. Accordingly, a method is provided for inhibiting fungal activity comprising contacting the pseudomycin prodrug of the present invention with a fungus. A preferred method includes inhibiting Candida albicans or Aspergillus fumigatus activity. The term “contacting” includes a union or junction, or apparent touching or mutual tangency of a compound of the invention with a fungus. The term does not imply any further limitations to the process, such as by mechanism of inhibition. The methods are defined to encompass the inhibition of parasitic and fungal activity by the action of the compounds and their inherent antifungal properties.

A method for treating a fungal infection which comprises administering an effective amount of a pharmaceutical formulation of the present invention to a host in need of such treatment is also provided. A preferred method includes treating a Candida albicans, Cryptococcus neoformans, or Aspergillus fumigatus infection. The term “effective amount” refers to an amount of active compound which is capable of inhibiting fungal activity. The dose administered will vary depending on such factors as the nature and severity of the infection, the age and general health of the host and the tolerance of the host to the antifungal agent. The particular dose regimen likewise may vary according to these factors. The medicament may be given in a single daily dose or in multiple doses during the day. The regimen may last from about 2-3 days to about 2-3 weeks or longer. A typical daily dose (administered in single or divided doses) contains a dosage level between about 0.01 mg/kg to 100 mg/kg of body weight of an active compound. Preferred daily doses are generally between about 0.1 mg/kg to 60 mg/kg and more preferably between about 2.5 mg/kg to 40 mg/kg. The host may be any animal including humans, companion animals (e.g., dogs, cats and horses), food-source animals (e.g., cows, pigs, sheep and poultry), zoo animals, marine animals, birds and the like.

### EXAMPLES

Unless indicated otherwise, all chemicals can be acquired from Aldrich Chemical (Milwaukee, Wis.).

### Biological Samples

P. syringae MSU 16H is publicly available from the American Type Culture Collection, Parklawn Drive, Rockville, Md., USA as Accession No. ATCC 67028. P. syringae strains 25-B1, 7H9-1, and 67 H1 were deposited with the American Type Culture Collection on Mar. 23, 2000 and were assigned the following Accession Nos.:

| | |
|---|---|
| 25-B1 | Accession No. PTA-1622 |
| 7H9-1 | Accession No. PTA-1623 |
| 67 H1 | Accession No. PTA-1621 |

### Chemical Abbreviations

The following abbreviations are used through out the examples to represent the respective listed materials:

ACN—acetonitrile

TFA—trifluoroacetic acid

DMAP—4-dimethylaminopyridine

DMF—dimethylformamide

EDCI—1-[3-(dimethylamino)propyl]-3-ethylcarbodiimide hydrochloride

BOC=t-butoxycarbonyl, (CH₃)₃C—O—C(O)—

CBZ=benzyloxycarbonyl, C₆H₅CH₂—O—C(O)—

PyBOP=benzotriazol-1-yloxy-tripyrrolidinophosphonium hexafluorophosphate

TBTU=o-Benzotriazol-1-yl-N,N,N′,N′-tetramethyluronium tetrafluoroborate

DIEA=N,N-diisopropylethylamine

The following structure II will be used to describe the products observed in Examples 1 and 2.

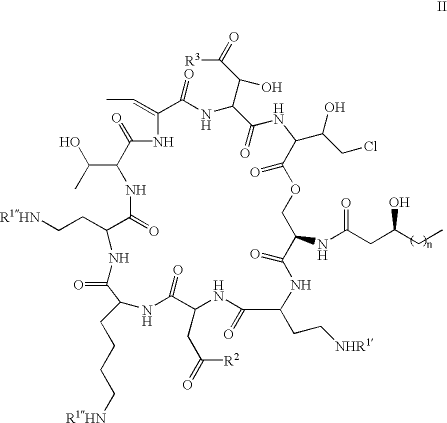

Detection and Quantification off Antifungal Activity:

Antifungal activity in the following examples were determined in vitro by obtaining the minimum inhibitory concentration (MIC) of the compound using a standard agar dilution test or a disc-diffusion test. A typical fungus employed in testing antifungal activity is Candida albicans. Antifungal activity is considered significant when the test sample (50 μl) causes 10-12 mm diameter zones of inhibition on C. albicans ×657 seeded agar plates.

Tail Vein Toxicity:

Mice were treated intravenously (IV) through the lateral tail vein with 0.1 ml of testing compound (20 mg/kg) at 0, 24, 48 and 72 hours. Two mice were included in each group. Compounds were formulated in 5.0% dextrose and sterile water for injection. The mice were monitored for 7 days following the first treatment and observed closely for signs of irritation including erythema, swelling, discoloration, necrosis, tail loss and any other signs of adverse effects indicating toxicity.

The mice used in the study were outbred, male ICR mice having an average weight between 18-20 g (available from Harlan Sprangue Dawley, Indianapolis, Ind.

In Vivo Testing Procedures:

The examples below were evaluated in a Mouse Model of Disseminated Candidiasis. Outbred, male ICR mice (average weight, 18-20 g; Harlan Sprangue Dawley, Indianapolis, Ind.) were used in a disseminated candidiasis ED₅₀ survival study. Candida albicans A26 was cultured on Sabouraud dextrose agar (SDA; DIFCO Laboratories; Detroit, Mich.) slants at 35° C. overnight. Blastoconidia were washed from the surface of the slant in sterile saline and quantitated using a hemacytometer. Mice were x-irradiated with 400 r 24 hr prior to infection with a Gamacell 40 (Atomatic Energy of Canada Limited Commercial Products, Ottawa, Canada). Mice were infected by an intravenous (IV) injection of 0.1 mL (containing 2×10⁶ blastoconidia per mouse) in the lateral tail vein. Untreated controls were moribund within 3-4 days post-infection. Mice were dosed four times at 0, 4, 24 and 48 hr post-infection with 0.2 mL of testing compounds which were given at 20, 10 and 5 mg/kg. Compounds were formulated in 4.0% hydroxypropyl cyclodextrin and sodium acetate, pH 7.0 buffer and 1.75% dextrose. Infected sham-treated mice (10 animals) were dosed with vehicle alone. Morbidity and mortality were recorded for 7 days. The 50% effective doses (ED₅₀) were determined using the method of Reed and Muench. Statistical differences in treated groups compared to untreated infection controls were determined using the Student's t test.

### Preparations

Preparation of (1a-1: R^{1a}=benzyl):

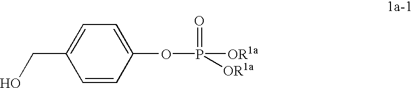

To a cooled (−20° C.) acetonitrile solution (31.5 mL) of 4-hydroxybenzyl alcohol (657 mg, 5.30 mmol) was added CCl₄ (2.5 mL, 26.5 mmol). The resulting milky solution was treated with EtPr₂N (1.9 mL, 11.13 mmol) and DMAP (65 mg, 0.53 mmol). After 10 min, the reaction mixture was treated with dibenzyl phosphite (1.39 g, 5.30 mmol) at −20° C. Upon completion, the reaction mixture was quenched with 0.5 N KH₂PO₄. The organic layer was separated and saved. The aqueous layer was extracted with EtOAc (3×10 mL). The combined organic layers were washed with brine and dried and concentrated in vacuo to give 2.27 g of the crude product 1a-1 in ˜100% yield.

Compounds where R^{1a}=—CH₃ (1a-2), R^{1a}=—CH₂CH₃ (1a-3) and R^{1a}=—CH₂CH₂Si(CH₃)₃ (1a-4) were also prepared using the same general procedures described above with the appropriate starting materials.

Preparation of (1b-1: R^{1a}=benzyl):

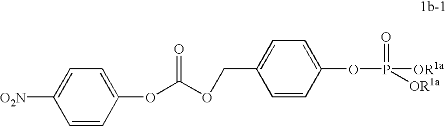

To a dichloromethane solution (12 mL) of the crude 1a-1 (2.27 g, 5.3 mmol) was added at 0° C. p-nitrophenyl chloroformate (1.18 g, 5.8 mmol). This was followed by pyridine (0.5 mL, 6.1 mmol). The reaction was stirred at 0° C. for 2 hr and quenched with saturated solution of NaHCO₃. The reaction mixture was extracted with EtOAc and the extract was washed with water and brine. The organic layer was dried and concentrated in vacuo to give an oily residue, which was purified with silica gel chromatography to afford 1.55 g (53%) of the desired product 1b-1.

Compounds where R^{1a}=—CH₃ (1b-2), R^{1a}=—CH₂CH₃ (1b-3) and R^{1a}=—CH₂CH₂Si (CH₃)₃ (1b-4) were also prepared using the same general procedures described above with the appropriate starting materials.

Preparation of (2a-1: R^{1b}=benzyl):

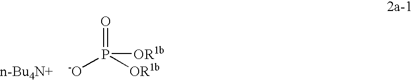

7.51 g (27 mmol) of dibenzyl phosphate was mixed with 17.5g (27 mmol) of 40% tetrabutyl ammonium hydroxide and the mixture was lyophilized to form an oil.

Compounds where R^{1b}=—CH₂CH₃ (2a-2) and R^{1b}=—CH₂CH₂CH₃ (2a-3) were also prepared using the same general procedures described above with the appropriate starting materials.

Preparation of (2b-1: R^{1b}=benzyl):

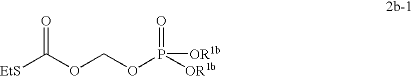

Compound 2a-1 (2.34 g, 4.3 mmol) was dissolved in 25 ml THF. S-ethyl-iodomethylthiocarbonate (1.14 g, 4.7 mmol) (prepared according to Folkmann, et al., Synthesis, 1159 (1990)) was added to the above solution at room temperature. The reaction was stirred at room temperature overnight. The mixture was filtered and the filtrates were concentrated in vacuo to give a brown oily residue, which was purified by column chromatography using 40% ethyl acetate in hexane to provide 0.86 g (51%) of 2b-1 as oil.

Compounds where R^{1b}=—CH₂CH₃ (2b-2) and R^{1b}=—CH₂CH₂CH₃ (2b-3) were also prepared using the same general procedures described above with the appropriate starting materials.

Preparation of (2c-1: R^{1b}=benzyl):

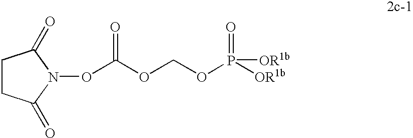

Compound 2b-1 (0.46 g, 1.1 mmol) was dissolved in 1 ml dichloromethane and cooled to −78° C. Sulfonylchloride (0.12 ml, 1.49 mmol) was added slowly. The mixture was stirred at −78° C. for 15 minutes and warmed slowly to room temperature. Stirring was continued at room temperature for three hours. The mixture was concentrated in vacuo to give the requisite chloroformate intermediate. The crude phosphate bearing chloroformate thus obtained was dissolved in 3 ml dichloromethane and cooled to 0° C. Ethyldipropylamine (0.3 ml) and 134 mg hydroxyl succinimide was added. The mixture was stirred at room temperature for 30 minutes to provide the crude 2c-1, which was used directly in Example 2.

Compounds where R^{1b}=—CH₂CH₃ (2c-2) and R^{1b}=—CH₂CH₂CH₃ (2c-3) were also prepared using the same general procedures described above with the appropriate starting materials.

### Example 1

The following example demonstrates the formation of mono-, di- and tri-substituted phosphate benzyloxycarbamate prodrugs of pseudomycin B (n=10, R² and R³=—OH).

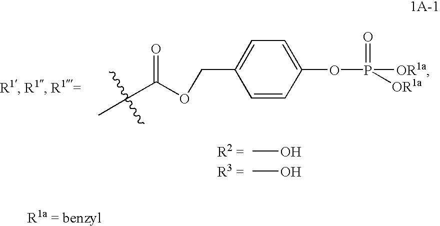

To a DMF solution (100 mL) of pseudomycin B (604 mg, 0.50 mmol) was treated with 1b-1 (0.909 g, 1.65 mmol) at room temperature. After stirring at room temperature for 24 hours, the reaction mixture was concentrated in vacuo, the resulting reaction mixture (containing mono-, di- and the desired triprodrug) was purified by reverse phase chromatography to afford 418 mg (34%) of the desired triprodrug 1A-1.

Compounds where R^{1a}=—CH₃ (1A-2), R^{1a}=—CH₂CH₃ (1A-3) and R^{1a}=—CH₂CH₂Si(CH₃)₃ (1A-4) were also prepared using the same general procedures described above with the appropriate starting materials.

The phosphate triester prodrug 1A-1 may be converted to the phosphoric acid monoester prodrug 1B via hydrogenation.

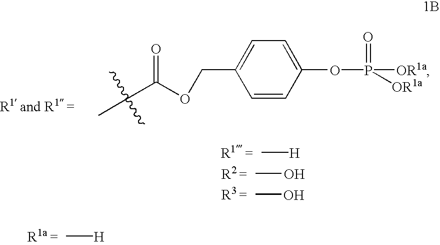

To a MeOH solution (10 mL) of 1A-1 (110.6 mg, 0.045 mmol) was added 10% Pd/C (60 mg) under nitrogen with caution. The reaction mixture was subjected to standard hydrogenation under 1.5 atm H₂. After 30 min, the reaction was stopped and the catalyst was filtered off. The filtrates were concentrated and diluted with 1:1 CH₃CN/H₂O and lyophilized to give 67.6 mg (89%) of product (mainly as diprodrug 1B as judged by its mass spectrum).

The ortho benzyl derivative (e.g., R¹=1(b) below) was synthesized using the same general procedures described above with the appropriate starting materials.

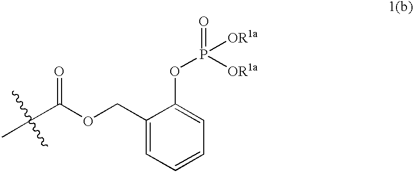

### Example 2

The following examples illustrates the formation of mono-, di- and tri-substituted phosphate and phosphoric acid monoester methyleneoxycarbamate prodrugs of pseudomycin B (n=10, R² and R³=—OH).

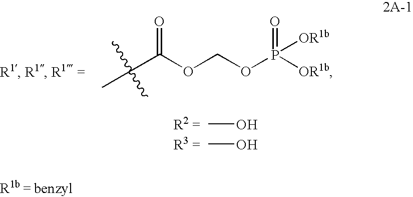

The crude 2c-1 was added to a solution of 300 mg (0.25 mmol) Pseudomycin B in 100 ml DMF. The mixture was stirred at room temperature overnight. The mixture was concentrated and purified by reverse phase preparative HPLC. 50 mg of 2A-1 (19%) was obtained.

Compounds where R^{1b}=—CH₂CH₃ (2A-2) and R^{1b}—CH₂CH₂CH₃ (2A-3) were also prepared using the same general procedures described above with the appropriate starting materials.

The phosphate triester prodrug 2A-1 may be converted to the phosphoric acid monoester prodrug 2B via hydrogenation.

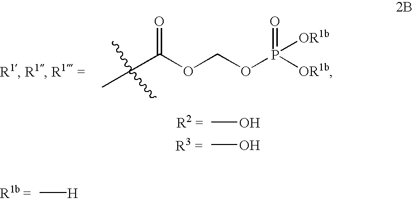

The purified product 2A-1 was dissolved 5 ml methanol. 50 mg of 10% Pd/C was added under nitrogen. Hydrogen was applied and the reaction mixture was stirred for one hour at room temperature. The mixture was filtered and the solvent was removed. The product was dissolved in water/acetonitrile (1/1) and lyophilized to give 32 mg (90% yield) of the desired product 2B.

An improvement in tail vein toxicity was observed in the phosphoric acid triester and monoester benxyloxycarbamate prodrugs (1A-1, 1A-2, 1A-3, 1A-4 and 1B) and the phosphoric acid triester and monoester methyleneoxycarbamate prodrugs (2A-1, 2A-2, 2A-3 and 2B) of pseudomycin B in comparison to pseudomycin B. Although both the para and ortho phosphoric acid monoester benzyloxycarbamate prodrugs showed in vivo activity, the best in vivo activity was observed with Compound 1B.

We claim:

1. A pseudomycin prodrug having the following structure: 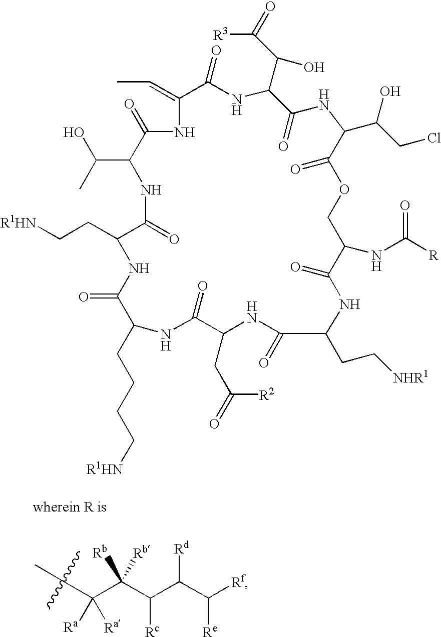
where
R^{a} and R^{a′} are independently hydrogen or 10 methyl, or either R^{a} or R^{a′} is alkyl amino, taken together with R^{b} or R^{b′} forms a six-membered cycloalkyl ring, a six-membered aromatic ring or a double bond, or taken together with R^{c} forms a six-membered aromatic ring;
R^{b} and R^{b′0} are independently hydrogen, halogen, or methyl, or either R^{b} or R^{b′} is amino, alkylamino, α-acetoacetate, methoxy, or hydroxy;
R^{c} is hydrogen, hydroxy, C₁-C₄ alkoxy, hydroxyalkoxy, or taken together with R^{e} forms a 6-membered aromatic ring or C₅-C₆ cycloalkyl ring;
R^{d} is hydrogen;
R^{e} is hydrogen, or taken together with R^{f} is a six-membered aromatic ring, C₅-C₁₄ alkoxy substituted six-membered aromatic ring, or C₅-C₁₄ alkyl substituted six-membered aromatic ring, and
R^{f} is C₉-C₁₈ alkyl, C₅-C₁₁ alkoxy or biphenyl;
or R is 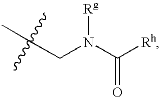
where
R^{g} is hydrogen, or C₁-C₁₃ alkyl, and
R^{h} is C₁-C₁₅ alkyl, C₄-C₁₅ alkoxy, (C₁-C₁₀ alkyl)phenyl, —(CH_{z})ₙ-aryl, or —(CH₂)ₙ-(C₅-C₆ cycloalkyl), where n=1 or 2; or
R is 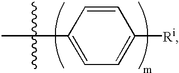
where
Rⁱ is a hydrogen, halogen, or C₅-C₈ alkoxy,
and m is 1, 2 or 3;
or R is 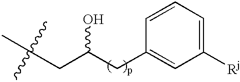
where
R^{j} is C₅-C₁₄ alkoxy or C₅-C₁₄ alkyl, and
p=0, 1 or 2:
or R is 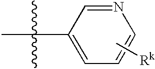
where
R^{k} is C₅-C₁₄ alkoxy; or
R is —(CH₂)-NR^{m}—(C₁₃-C₁₈ alkyl), where R^{m} is H, —CH₃ or —C(O)CH₃;
R^{l} is independently hydrogen or a group represented by formula 1(a), 1(b), or 1(c) 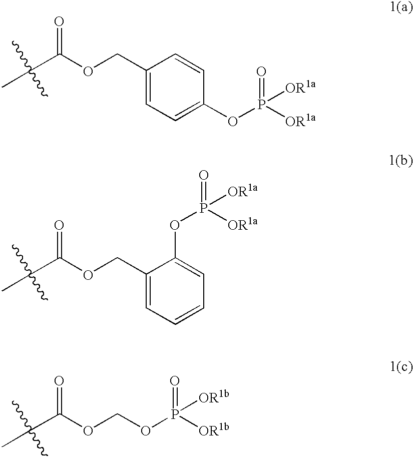
where
R^{1a} is hydrogen, C₁-C₆ alkyl, benzyl, or —CH₂CH₂Si(CH₃)₃, and
R^{1b} is hydrogen or C₁-C₆ alkyl, provided that at least one R^{l} is a group represented by formula 1(a), 1(b) or 1(c);
R² and R³ are independently —OR^{2a} or —N(R^{2b})(R^{2c}),
where
R^{2a} and R^{2b} are independently hydrogen, C₁-C₁₀ alkyl, C₃-C₆ cycloalkyl, hydroxy(C₁-C₁₀ ) alkyl, alkoxy(C₁-C₁₀ )alkyl, C₂-C₁₀ alkenyl, amino(C₁-C₁₀ )alkyl, mono- or di-alkylamino(C₁-C₁₀ )alkyl, aryl (C₁-C₁₀ ) alkyl, heteroaryl (C₁-C₁₀ ) alkyl, cycloheteroalkyl (C₁-C₁₀ ) alkyl, or
R^{2b} is an alkyl carboxylate residue of an aminoacid alkyl ester and R^{2c′} is hydrogen or C₁-C₆ alkyl: and
pharmaceutically acceptable salts and solvates thereof.

2. The prodrug of claim 1 wherein R¹ is represented by structure 1(a): 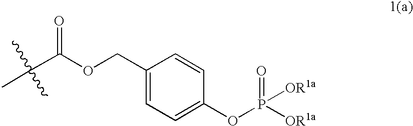
where R^{1a} is hydrogen, C₁-C₆ alkyl, benzyl, or —CH₂CH₂Si(CH₃)3.

3. The prodrug of claim 2 wherein R^{1a} is hydrogen.

4. The prodrug of claim 1 wherein R¹ is represented by structure 1(b): 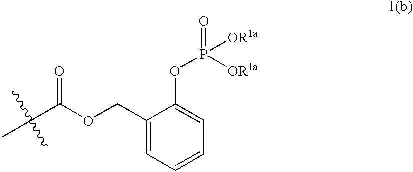
where R^{1a} is hydrogen, C₁-C₆ alkyl, benzyl, or —CH₂CH₂Si(CH₃)₃.

5. The prodrug of claim 4 wherein R^{1a} is hydrogen.

6. The prodrug of claim 1 wherein R1 is represented by the structure 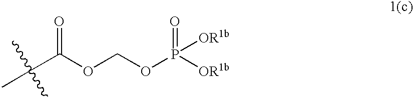
where R^{1b} is hydrogen or C₁-C₆ alkyl.

7. The prodrug of claim 2 wherein R is represented by the structure 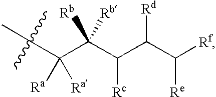
where R^{b′} is hydroxy, R^{a}, R^{a′}, R^{b}, R^{c}, R^{d}, and R^{e} are all hydrogen, and R^{f} is n-octyl.

8. The prodrug of claim 3 wherein R is represented by the structure 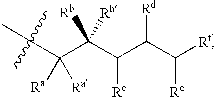
where R^{b′} is hydroxy, R^{a}, R^{a′}, R^{b}, R^{c}, R^{d}, and R^{e} are all hydrogen, and R^{f} is n-octyl.

9. The prodrug of claim 4 wherein R is represented by the structure 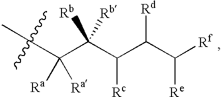
where R^{b′} is hydroxy, R^{a}, R^{a′}, R^{b}, R^{c}, R^{d}, and R^{e} are all hydrogen, and R^{f} is n-octyl.

10. The prodrug of claim 5 wherein R is represented by the structure 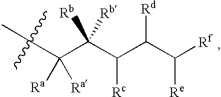
where R^{b′} is hydroxy, R^{a}, R^{a′}, R^{b}, R^{c}, R^{d}, and R^{e} are all hydrogen, and R^{f} is n-octyl.

11. The prodrug of claim 6 wherein R is represented by the structure 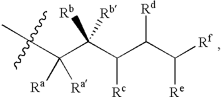
where R^{b′} is hydroxy, R^{a}, R^{a′}, R^{b}, R^{c}, R^{d}, and R^{e} are all hydrogen, and R^{f} is n-octyl.

12. The prodrug of claim 1 wherein said alkyl carboxylate residue of an aminoacid alkyl ester is represented by —CH₂CO₂CH₃, —CH(CO₂CH₃)CH(CH₃)₂, —CH(CO₂CH₃)CH(phenyl), —CH(CO₂CH₃)CH₂OH, —CH(CO₂CH₃)CH₂(p-hydroxyphenyl), —CH(CO₂CH₃)CH₂SH, —CH(CO₂CH₃)CH₂(CH₂)₃NH₂, —CH(CO₂CH₃)CH₂(4-imidazole), —CH(CO₂CH₃)CH₂(5-imidazole), —CH(CO₂CH₃)CH₂CO₂CH₃, or —CH(CO₂CH₃)CH₂CO₂NH₂.

13. A pseudomycin prodrug having the following structure: 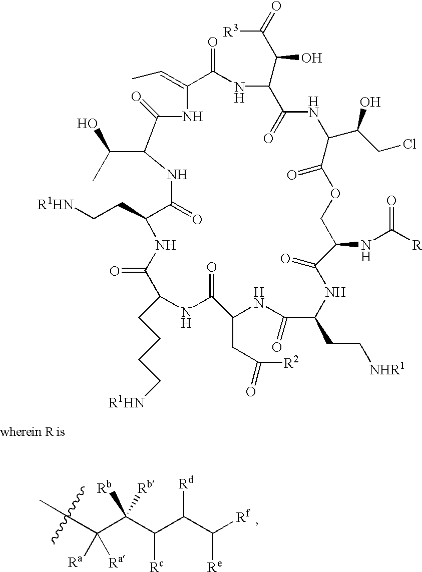
where
R^{a} and R^{a} are independently hydrogen or methyl, or either R^{a} or R^{a′} is alkyl amino, taken together with R^{b} or R^{b′} forms a six-membered cycloalkyl ring, a six-membered aromatic ring or a double bond, or taken together with R^{c} forms a six-membered aromatic ring;
R^{b} and R^{b′} are independently hydrogen, halogen, or methyl, or either R^{b} or R^{b′} is amino, alkylamino, α-acetoacetate. methoxy, or hydroxy;
R^{c} is hydrogen, hydroxy, C₁-C₄ alkoxy, hydroxyalkoxy, or taken together with R^{e} forms a 6-membered aromatic ring or C₅-C₆ cycloalkyl ring;
R^{d} is hydrogen;
R^{e} is hydrogen, or taken together with R^{f} is a six-membered aromatic ring, C₅-C₁₄ alkoxy substituted six-membered aromatic ring, or C₅-C₁₄ alkyl substituted six-membered aromatic ring, and
R^{f′} is C₈-C₁₈ alkyl, C₅-C₁₁ alkoxy or biphenyl;
or R is 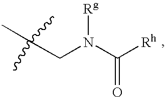
where
R^{g} is hydrogen, or C₁-C₁₃ alkyl, and
R^{e} is C₁-C₁₅ alkyl, C₄-C₁₅ alkoxy, (C₁-C₁₀ alkyl)phenyl, —(CH₂)ₙ-aryl, or —(CH₂)ₙ-(C₅-C₆ cycloalkyl), where n=1 or 2; or
R is 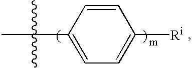
where
Rⁱ is a hydrogen, halogen, or C₅-C_{B} alkoxy, and m is 1, 2 or 3;
or R is 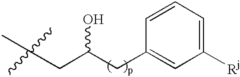
where
R^{j} is C₅-C₁₄ alkoxy or C₅-C₁₄ alkyl, and
p=0, 1 or 2;
or R is 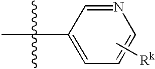
where
R^{k} is C₅-C14 alkoxy; or
R is —(CH₂)—NR^{m}—(C₁₃-C₁₈ alkyl), where R^{m} is H, —CH₃ or —C(O) CH₃;
R^{l} is independently hydrogen or a group represented by formula 1(a), 1(b), or 1(c) 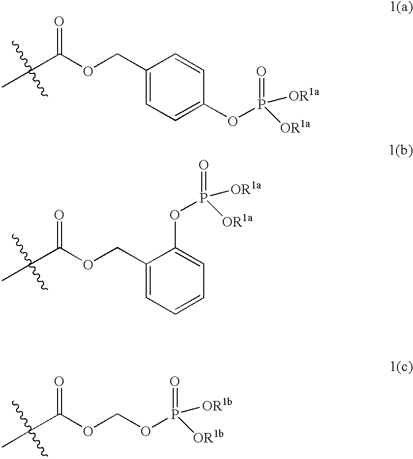
where
R^{1a} is hydrogen, C₁-C₆ alkyl, benzyl, or —CH₂CH₂Si(CH₃)₃, and
R^{1b} is hydrogen or C₁-C₆ alkyl, provided that at least one R¹ is a group represented by formula 1(a), 1(b) or 1(c);
R² and R³ are independently —OR^{2a} or —N(R^{2b})(R^{2c}),
where
R^{2a} and R^{2b} are independently hydrogen, C₁-C₁₀ alkyl, C₃-C₆ cycloalkyl, hydroxy(C₁-C₁₀)alkyl, alkoxy(C₁-C₁₀)alkyl, C₂-C₁₀ alkenyl, amino(C₁-C₁₀)alkyl, mono- or di-alkylamino(C₁-C₁₀)alkyl, aryl (C₁-C₁₀) alkyl, heteroaryl (C₁-C₁₀) alkyl, cycloheteroalkyl(C₁-C₁₀)alkyl, or
R²b is an alkyl carboxylate residue of an aminoacid alkyl ester and R^{2c} is hydrogen or C₁-C₆ alkyl; and
pharmaceutically acceptable salts and solvates thereof.

14. The prodrug of claim 13 wherein R^{l} is represented by structure 1(a): 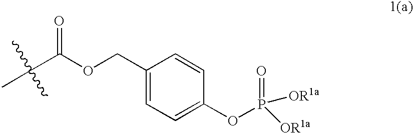
where R^{1a} is hydrogen, C₁-C₆ alkyl, benzyl, or —CH₂CH₂Si(CH₃)₃.

15. The prodrug of claim 14 wherein R^{1a} is hydrogen.

16. The prodrug of claim 13 wherein R^{l} is represented by structure 1(b): 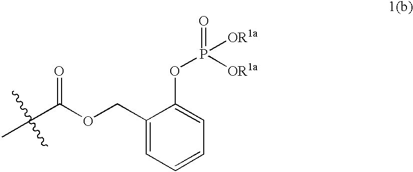
where R^{1a} is hydrogen, C₁-C₆ alkyl, benzyl, or —CH₂CH₂Si(CH₃)₃.

17. The prodrug of claim 16 wherein R^{1a} is hydrogen.

18. The prodrug of claim 13 wherein R1 is represented by the structure 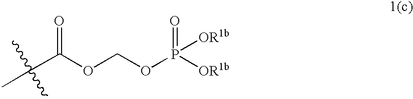
where R^{1b} is hydrogen or C₁-C₆ alkyl.

19. The prodrug of claim 15 wherein R is represented by the structure 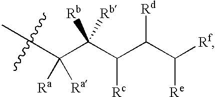
where R^{b′} is hydroxy, R^{a}, R^{a′}, R^{b}, R^{c}, R^{d}, and R^{e} are all hydrogen, and R^{f} is n-octyl.

20. The prodrug of claim 17 wherein R is represented by the structure 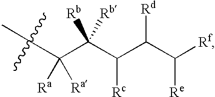
where R^{b′} is hydroxy, R^{a}, R^{a′}, R^{b}, R^{c}, R^{d}, and R^{e} are all hydrogen, and R^{f} is n-octyl.

21. The prodrug of claim 18 wherein R is represented by the structure 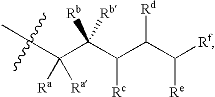
where R^{b′} is hydroxy, R^{a}, R^{a′}, R^{b}, R^{c}, R^{d}, and R^{e} are all hydrogen, and R^{f} is n-octyl.

22. The prodrug of claim 13 wherein said alkyl carboxylate residue of an aminoacid alkyl ester is represented by —CH₂CO₂CH₃, —CH(CO₂CH₃)CH(CH₃)₂, —CH(CO₂CH₃)CH(phenyl), —CH(CO₂CH₃)CH₂OH, —CH(CO₂CH₃)CH₂(p-hydroxyphenyl), —CH(CO₂CH₃)CH₂SH, —CH(CO₂CH₃)CH₂(CH₂)₃NH₂, —CH(CO₂CH₃)CH₂(4-imidazole), —CH(CO₂CH₃)CH₂(5-imidazole), —CH (CO₂CH₃)CH₂CO₂CH₃, or —CH(CO₂CH₃)CH₂CO₂NH₂.

23. A pharmaceutical formulation comprising a pseudomycin prodrug of Claim 8, 10, 19 or 20 and a pharmaceutically acceptable carrier.

24. A medicament for treating a fungal infection in an animal wherein said medicament comprises a compound of claims 8, 10, 19 or 20.

25. A method for treating a fungal infection in an animal in need thereof, comprising administering to said animal a pseudomycin prodrug of claims 8, 10, 19 or 20.
